(19) United States
(12) Patent Application Publication
(10) Pub. No.: US 2007/0293579 A1
(43) Pub. Date: Dec. 20, 2007

(54) USE OF CARP INHIBITORS FOR THE TREATMENT OF HEART DISEASES

(76) Inventors: Franz-Werner Kluxen, Muehltal (DE); Bernd Hentsch, Frankfurt (DE); Claudia Wilm, Darmstadt (DE); Marian Braendle, Starzach (DE); Thomas Ehring, Remscheid (DE); Thomas Eschenghagen, Hamburg (DE); Oliver Zolk, Dueren (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/676,990

(22) Filed: Feb. 20, 2007

Related U.S. Application Data
(63) Continuation of application No. 10/363,903, filed on Aug. 7, 2003.

(30) Foreign Application Priority Data
Nov. 9, 2000 (EP) 0119771.4

(51) Int. Cl. A61K 31/145; A61P 9/00; C12Q 1/68; G01N 33/50

(52) U.S. Cl. 514/649; 435/6; 435/7.1

(57) ABSTRACT

The present invention relates to the novel finding that inhibition of human CARP protein or of CARP DNA/mRNA may successfully cure heart disorders, especially heart failure in humans. The invention relates, therefore, to pharmaceutical compositions containing substances, preferably of lower molecular weight, which may influence the activity of cardiac CARP. The invention relates, furthermore, to a method of treatment heart failure using CARP inhibitory compounds, to methods for screening these inhibitors and to the use of CARP as diagnostic tool.

The present invention relates to the novel finding that inhibition of human CARP protein or of CARP DNA/RNA may successfully cure heart disorders, especially heart failure in humans. The invention relates, therefore, to pharmaceutical compositions containing substances, preferably of lower molecular weight, which may influence the activity of cardiac CARP. The invention relates, furthermore, to a method of treatment heart failure using CARP inhibitory compounds, to methods for screening these inhibitors and to the use of CARP as diagnostic tool.

### BACKGROUND OF THE INVENTION

Human CARP (cardiac-restricted ankyrin repeat protein), a nuclear protein predominantly expressed in cardiac myocytes was first detected by Chu, W. et al., 1995 (The Journal of Biological Chemistry 270, 10236-10245) as a mRNA that is strongly upregulated in human endothelial cells upon induction of the cells by interleukin-1α or TN F-α. The authors could show that CARP is a nuclear localized protein having a molecular weight of 36,000 and that the protein is able to bind to DNA and acts as a transcription factor. It was found that CARP is not expressed in a variety of cells of different origins (epithelial cells, bladder carcinoma cells, fibroblasts, melanoma cells, and cells of hematopoietic origin). However, in fibroblast cells, CARP mRNA expression is as well induced by treatment of the cells with TNF-α.

Later Zou et al., 1997 (Development 124, 793-804) identified the rat homologue of CARP during a search for heart specific transcription factors. They had previously identified a promoter element, that confers ventricular specific expression of genes (HF-1a/MEF-2) and an ubiquitous expressed transcription factor, YB-1 that binds to this element. They used YB-1 as a bait in a yeast two hybrid screen and found CARP. Subsequently they showed that CARP mRNA expression is very restricted and most prominent in the heart. In a number of experiments they could show, that CARP and YB-1 physically form dimers in vivo. In addition, it was found that CARP itself is regulated by the NKx2.5 homeobox gene. Co-transfection experiments demonstrated that that CARP down regulates genes that are under control of the HF-1a elements in cardiac myocytes as well as in non myocytes (Ross, R. S. et al., 1996, Development 122, 1799-1809). Jeyaseelan et al., 1997 (The Journal of Biological Chemistry 272, 22800-22808) found that CARP is able to down regulate transcription from the ANF promoter and the cTNC promoter in cells from cardiac and non cardiac origin. With respect to the ANF promoter, they showed that CARP acts antagonistic to the NkX2.5 gene, which strongly induces transcription for this promoter.

Baumeister et al., 1997 (The Journal of Cell Biology 139, 1231-1242) found CARP as a protein that is highly induced in skeletal muscle by denervation. In addition, they showed that CARP is upregulated in the hearts of MLP-deficient (muscle LIM protein) mice. These mice develop a dilated cardiomyopathy and hypertrophy. They speculated that CARP might be an immediate early gene: the rat, mouse and human mRNA have ATTTA degradation motifs in the 3′ UTR and the protein has a PEST-like sequence which is characteristic for rapidly degraded proteins. However, in the denervated muscle, CARP expression is elevated for at least 7 days (the longest period examined).

CARP was found to be upregulated in the hearts of doxorubicin treated animals (rabbits and rats, Aihara, Y. et al., 1999, Biochimica et Biophysica Acta 1447, 318-324) indicating that CARP might play a role in this pathology. Kuo et al., 1999 (Development 126, 4223-4234) used a mouse model of cardiac hypertrophy (transverse aortic constriction) and found that CARP mRNA is strongly induced 4 and 7 days after the surgery. They also showed that ANF mRNA is elevated as well. This finding is somewhat contradictory to the finding of Jeyaseelan et al. (I.c.) who found that CARP down regulated the transcription from the ANF promoter. This may be due to (i) CARP and ANF transcripts being elevated in different cells of the organ or (ii) the ANF promoter construct used by Jeyaseelan et al. (700 bp) did not contain all the genetic elements that regulate the expression of ANF mRNA or (iii) the CARP inhibition of ANF mRNA expression is overridden by yet an unknown mechanism.

### GENERAL DESCRIPTION OF THE INVENTION

This invention discloses experiments which verify the finding that in some models of cardiac pathology, the expression of CARP mRNA is elevated and extend this to human pathology. This is the first description that, in addition to the CARP mRNA, the CARP protein is as well up-regulated in comparison to healthy control hearts. Thus, it is well established that CARP may play a role in pathological conditions of the human heart. It is yet to be established, whether the elevated CARP expression is causative for the disease, or is an adaptive process of the cells in response to the pathological situation.

There are numerous publications which show that in human heart failure as well as in animal models of heart failure and heart hypertrophy embryonic forms of e.g. myofibrillar proteins a expressed and replace the adult forms. CARP is known to play a role in the regulation of the switch from fetal to adult forms of the contractile proteins like cTNT. CARP is a repressing transcription factor inhibiting the expression of the adult forms. In addition, it has been described that CARP inhibits the expression of ANF, which as well is a hallmark of later stages of heart failure disease.

The role of CARP in fibroblasts deserves a more close inspection. Jeyseelan et al., (I.c.) claim that CARP mRNA is not expressed in cardiac fibroblasts and is not inducible in these cells by treatment with TNF-α or LPS. In contrast, it Was described that, although CARP is not expressed in the fibroblast cell line MRC-5, it is easily induced in these cells by exposing them to TNF-α. Some preliminary experiments have been conducted to clarify the cellular distribution of CARP mRNA in the normal rat heart. Our in situ hybridization results using CARP antisense cRNA suggest a expression in fibroblast like cells and not in cardiomyocytes. This is in clear contrast to most of the published data and demands for a more detained analysis.

It is concluded that the inhibition of the CARP function may help to restore the adult phenotype of the contractile proteins mRNA and may as well help to increase the expression of ANF protein. In comparison to other transcription factors that are involved in regulation of these genes, like YB-1, CARP is almost exclusively expressed in the heart and only at very little amounts in other tissues, namely skeletal muscle. This makes a pharmacological intervention towards CARP very attractive since only minor side effects may be observed, if any.

The present invention investigated whether chronic beta-adrenergic stimulation, which is known to induce cardiac hypertrophy, alters CARP-expression in vivo and in vitro. In the affirmative case it would be an object of this invention to provide inhibitors of CARP protein or CARP mRNA for the manufacture of a medicament applicable for the treatment of heart diseases.

The invention shows that CARP is significantly upregulated in human heart failure. In vitro and in vivo studies according to this invention suggest that CARP acts as a negative regulator of alpha-MHC gene expression. Increased CARP levels in human heart failure may contribute to altered gene expression and contractile function. CARP is increased during ISO-induced cardiac hypertrophy in vivo and is increased by direct beta-adrenoceptor stimulation of cardiac myocytes in vitro. The ERK/p42/p44 MAP kinase pathway seems to be involved in the signaling process.

Thus, is an object of this invention to provide inhibitors of CARP which can be used in the treatment of heart diseases, preferably heart failure or heart hypertrophy. One of these inhibitors, suitable for blocking CARP expression is the ERK inhibitor family (e.g. U0126).

In detail, the invention discloses the following issues:
use of an inhibitor or antagonist of CARP protein or CARP mRNA for the manufacture of a medicament which can be used for the treatment of heart diseases;
a corresponding use, wherein the heart disease is heart failure or heart hypertrophy;
a corresponding use, wherein the heart hypertrophy is induced by isoprenalin or phenylephrine;
a corresponding use, wherein the inhibitor is capable of binding to CARP protein and/or CARP mRNA;
a corresponding use, wherein the inhibitor prevents CARP protein from binding to YB-1;
a corresponding use, wherein the inhibitor prevents CARP/YB-1 complex from binding to HF-1a/HF1-b/Mef-2 DNA elements;
a corresponding use, wherein the inhibitor prevents CARP protein from down-regulation of ANF or TNC from cardiac origin;
a corresponding use, wherein the inhibitor is capable of enhancing the expression and/or secretion of ANF from the heart;
a pharmaceutical composition applicable for the treatment of heart diseases comprising a substance having at least one of the following biological properties:
(i) inhibition CARP protein and/or CARP mRNA,
(ii) binding to CARP protein and/or CARP mRNA,
(iii) prevention of CARP from binding to YB-1, or of CARP/YB-1 complex from binding to HF-1a/HF1-b/Mef-2 DNA elements,
(iv) prevention of CARP protein from down-regulation of cardiac ANF or TNC,
(v) enhancement of the expression and/or secretion of ANF from the heart;
a use of CARP Protein or CARP mRNA/DNA for screening of substances which block or inhibit CARP or CARP expression;
a use of CARP protein or CARP mRNA/DNA as negative regulator of the expression of alpha-myosin heavy chain mRNA;
a method for screening an inhibitor of CARP protein and/or CARP mRNA, the method comprising the following steps
(i) constructing a first expression vector comprising full-length human CARP cDNA together with a strong promoter and a selection markers
(ii) constructing a second expression vector comprising a reporter gene which is under the control of HF-1a/HF1b/Mef-2 together with a selection marker which is different from that of the first expression vector
(iii) transfecting said first and said second expression vector into a suitable eukaryotic expression cell line, and
(iv) culturing transfected cells expressing CARP protein as well the reporter gene product which indicates the CARP activity, together with said inhibitor and measuring the reduced activity of said reporter gene product;
a use of CARP or modifications or variants of CARP having the biological properties of CARP as diagnostic means for detecting in vitro heart disorders like heart failure; and finally
a method of treating heart diseases, wherein the method comprises administering to a patient suffering from heart failure a pharmaceutical composition as described above in an amount, which is effective for a specific heart disease, preferably heart failure and hypertrophy.

The following examples describe the invention in more detail.

### EXAMPLE 1

### Northern Blot

CARP is believed to play a role in human pathology as well. To analyze the expression of CARP mRNA in human hearts, northern blots using a human CARP cDNA were performed. mRNAs for the northern blot were derived from human hearts of patients with chronic heart failure (stage NYHA III-IV) due to idiopathic dilated cardiomyopathy (DCM, n=11) or to ischemic cardiomyopathy (ICM, n=12). As controls, non-failing donor hearts were used (n=11). The northern blots were normalized by the analysis of GAPDH.

Results (Arbitrary Units):

| | Non-failing | DCM | ICM |
|---|---|---|---|
| CARP mRNA/ | 0.67 ± 0.14 | 1.1 ± 0.09 | 1.1 ± 0.08 |
| GAPDH mRNA | n = 11 | N = 11 | n = 12 |

These results clearly show, that CARP mRNA is significantly upregulated in the hearts of patients suffering from DCM or ICM.

### EXAMPLE 2

### Antibody Production

First, recombinant CARP for the immunization was produced. For this purpose, CARP mRNA was inserted into an inducible E. coli expression vector. From the inclusion bodies, CARP was solubilized in 1-5 M Guanidine-HCl and CARP protein purified by several chromatographic steps. Finally, the Guanidine-HCl was removed by dialysis against PBS. CARP protein was used to immunize 2 rabbits. An CARP antiserum was collected from the animals and tested for activity. The antiserum was analyzed by an ELISA assays and was found to have a reasonable titer of antibodies directed against CARP.

The antibody was also tested in Western Blots using purified CARP and protein extracts from human and rat heart. It was found that the sample proteins had to be reduced with DTT and subsequently carboxymethylated by incubation with 0.5M Iodoacetamid. This was necessary since CARP has several cysteine residues which have a high tendency to form inter- and intramolecular covalent bonds. This leads to the formation of dimers and tetramers and to the detection of multiple bands in gel electrophoresis and western blot experiments. When the above treatment is applied to the samples, in western blots a single band of about 35 kDa is detected. This size is in agreement with the size calculated from the amino acid sequence of CARP.

### EXAMPLE 3

### Immunological Analysis of CARP Protein Expression in Normal and Diseased Tissues

Next, the expression of the CARP protein was analyzed to show that the protein levels reflect the elevated CARP mRNA levels. For this purpose, protein samples derived from human non-failing, DCM, and ICM hearts were analyzed. The western blots were normalized by analysis of calsequestrin. The following results have been obtained:

| | Non-failing | DCM | ICM |
|---|---|---|---|
| CARP/ | 0.62 ± 0.13 | 1.38 ± 0.20 | 1.71 ± 0.31 |
| calsequestrin | n = 9 | N = 9 | n = 9 |

These results clearly show that CARP protein is as well elevated in the pathological human hearts. Moreover, the protein is even more elevated in comparison to the control hearts than the CARP mRNA. This may be due to a higher turnover rate of the mRNA than the protein.

### EXAMPLE 4

### Use of CARP Derived Tools as a Diagnostic Tool

CARP cDNA/cRNA

CARP cDNA and/or cRNA may be used for the detection of elevated CARP mRNA levels in cardiac biopsies from hearts of patients at risk. The CARP expression status may as well be used for the analysis of human hearts that will be used for transplantation to ensure the healthy status of that heart. CARP mRNA level may be measured by any hybridization based method like northern blot, dot blot, RNAse protection or similar methods relying on the hybridization of CARP cDNA or cRNA to the target RNA derived from the tissues to be analyzed.

CARP Antibodies

Antibodies directed against human native of recombinant CARP may be used for diagnostic purposes as well. The amount of CARP protein in biopsies from hearts of patients at risk can be measured by an ELISA, RIA, western blot or similar techniques. An elevated level of CARP protein may indicate a pathological status of the heart analyzed. In addition, hearts scheduled for transplantation may also be analyzed for their status by any of the antibody based methods described in this section.

CARP PCR

CARP cDNA derived sequences may be used for the design of primers that can be applied for any PCR based method for the quantitation of CARP mRNA. The quantitation of CARP mRNA levels in biopsies from hearts of patients at risk for heart failure may be used as a diagnostic tool to detect altered gene expression associated with heart failure. This method can also be applied to hearts used for transplantation.

### EXAMPLE 5

### Screening of CARP Inhibiting Substances

Stable transfection assay: The use of human CARP cDNA for the construction of cell lines for screening of compounds that interfere with the biological action of human CARP. Such assays may be configured as follows: the full length human CARP cDNA is inserted in a suitable eukaryotic expression vector (under control of a strong promoter like SV40, CMV, or an inducible promoter (e.g. test on-off system)) carrying a selection marker like neomycin, zeozin, hygromycin, or other substances for selection of recombinant cells. This expression vector is transfected by standard methods into the cell line h9c2 and selected for a stable cell line by adding the antibiotic. In this way, either a monoclonal or a polyclonal cell line is selected. Next, an eukaryotic expression plasmid containing a reporter gene like luciferase, β-galactosidase, alkaline phosphatase or similar easy detectable gene controlled by a HF-1a/HF1b/Mef-2 as described in Ross et al. is transfected into this cell line. This second plasmid should contain a selection maker different from the one used to construct the CARP cell line. In this ways a cell line containing two genes is constructed: one gene constitutively expresses CARP and the second is a reporter plasmid detecting the activity of CARP. This cell line is used for the detection of test substances interfering with the activity of CARP. For this purpose, the cell line is grown in suitable MTP plates and substances are added. After an incubation of 6-12 hours, substances that interfere with the biological activity of CARP can be detected be measuring the activity of the reporter gene. When the activity of the substance interferes with the action of CARP, a reduced signal is measured.
Stable transfection assay: The plasmids used in section 1. may also be used in other standard cell lines like COS-1, COS-7, HEK293, HEK293 EBNA, CHO, BHK, HeLA or similar cell lines for the expression of cDNAs.
Transient transfection assay: The assays described in section 1. and 2. may be modified like this: the plasmids carrying CARP and the reporter gene are mixed and are transfected using standard techniques in a cell line described in sections 1. or 2. The test substances are added and after 24-72 h the activity of the reporter gene is measured. Substances that interfere with the CARP activity can be detected by a lower activity of the reporter gene than in comparison to the non-treated control cells.
CARP—YB-1 binding assay (i): CARP is known to interact with the ubiquitous transcription factor YB-1. A full length or partial cDNA encoding YB-1 is inserted into a vector allowing the expression of YB-1 with an N-terminal or an C-terminal His-Tag, or a GST protein. Other genes or amino acid sequences that allow for the detection and/or purification of the recombinant fusion protein may apply as well. The fusion protein is expressed in E. coli, or in a Bakulovirus/Sf9 system, or in an in vitro translation system. Next, the purified or crude fusion protein is bound to a suitable matrix depending on the tag used. Test substances are added and allowed to bind to YB-1. Purified CARP is added and allowed to bind to the YB-1 fusion protein. Unbound CARP is washed away and the remaining CARP is detected using an antibody directed against human recombinant CARP. Test substances that interfere with the binding of CARP to YB-1 are detected by a lowered signal.
CARP—YB-1 binding assay (ii): CARP and YB-1 may be used in a yeast two hybrid screen. CARP or YB-1 are used for the construction of the DNA-binding and the trans-activation domain fusion protein respectively, or vice versa. A standard commercial yeast two hybrid system may be used. After the construction of the recombinant yeast strains, test substances may be screened for interference with the YB-1/CARP interaction.
CARP—YB-1 binding assay (iii): CARP and YB-1 may be used in a mammalian two hybrid screen. CARP or YB-1 are used for the construction of the DNA-binding and the activation domain fusion protein respectively, or vice versa. A standard commercial mammalian two hybrid system may be used. After the construction of the recombinant mammalian cell line, test substances may be screened for interference with the YB-1 (CARP interaction.
CARP—YB-1 binding assay (vi): Recombinant purified CARP and YB-1 proteins may be used for a protein-protein interaction assay. For this purpose, one of the two proteins is immobilized either on a modified surface of the reaction vessel or by binding to a suitable matrix when the protein is expressed as a fusion protein. The other of the two proteins is labeled with ¹²⁵I or labeled with ³⁵S when produced by in vitro translation of it's mRNA or cRNA. The labeled protein is incubated with the immobilized protein and, after washing away non-bound protein, the binding is detected by measuring the radioactivity. When a substance is added that interferes with the binding of the two proteins, a lower amount of radioactivity is measured. As an alternative, the labeled protein may be labeled with a fluorescent dye and the binding may be detected by measuring the remaining fluorescence after washing. The binding may as well be detected by measuring the change in fluorescence polarization. The proteins may also be labeled by europium cryptate and a fluorescent dye or similar dyes to detect the binding event fluorescence resonance energy transfer (FRET). These kinds of assays are homogenous and do not need an washing step. The binding event may as well be measured by electrochemiluminescence. For this purpose, one of the proteins may be immobilized on magnetic beads by a tag like His(6) or GST or similar. The other protein is labeled by ruthenium tris-bipyridyl compound (Ru(bpy)₃²⁺). The binding is detected on a electrode that has captured the magnetic bead carrying the protein complex.

### EXAMPLE 6

### CARP Expression and Purification

CARP cDNA can be inserted in vectors suitable for expression of the protein in E. coli, Sf9 cells, or eukaryotic cells as described in standard literature like e.g. The cDNA may be modified by a tag like His(6), GST, FLAG, or similar to aid the purification of the protein.

### EXAMPLE 7

Subtractive hybridization techniques, aimed at identifying target genes for drug development, showed an upregulation of cardiac ankyrin protein (CARP) mRNA in experimental canine heart failure.

Here CARP expression was determined in myocardium from non-failing donor hearts (NF) and hearts from patients with dilated (DCM) or ischemic cardiomyopathy (ICM) by Northern and Western blot analysis. mRNA (CARP/GAPDH mRNA NF: 0.7±0.14, n=11; DCM: 1.2±0.09, n=11; ICM: 1.1±0.08, n=12) and protein levels (related to calsequestrin expression; NF: 1.0±0.23, n=11; DCM: 1.8±0.12, n=11; ICM: 1.8±0.20, n=8) were significantly increased in left ventricular myocardium from patients with heart failure irrespective of etiology. In atrial myocardium, no significant difference in CARP expression was observed between failing and non-failing hearts (n=8). To gain further insights into the function of CARP, an adenoviral vector containing human CARP cDNA (Ad. CARP) was generated. Infection of cardiac myocytes isolated from neonatal rats with Ad. CARP at increasing MOI caused a dose related overexpression of CARP. To address the question whether CARP acts as a regulator of sarcomeric protein gene expression, mRNA concentrations of actin and myosin heavy chain (MHC) isoforms were determined by quantitative RT-PCR. Alpha-MHC mRNA levels in myocytes infected with Ad. CARP (MOI 50) were decreased to 25% of mRNA concentrations in myocytes infected with control virus (n=10, p<0.05).

### EXAMPLE 8

Male Wistar rats were treated with isoprenaline (ISO, 2.4 mg/kg/d, Alzet mini pump) for 4 days. Controls were infused with NaCl. ISO treated rats exhibited a marked cardiac hypertrophy with a 40% increase in heart weight/body weight ratio. In ISO treated rats CARP mRNA expression, as assessed by Northern blot analysis, was increased in the left ventricular (LV) myocardium by 140% (CARP mRNA/GAPDH mRNA: ISO 3.1±0.5, n=6; NaCl: 1.3±0.12, n=11; p<0.05). Western blot analysis confirmed a 50% increase in CARP protein (CARP/calsequestrin: ISO 0.61±0.07; n=6; NaCl: 0.41±0.03, n=10; p<0.05). To gain further insight into the mechanism by which CARP expression is regulated, isolated myocytes were treated with ISO (1 μM) and phenylephrine (PE, 50 μM). For comparison the effects of the alpha-adrenoceptor agonist phenylephrine (PE, 50 μM) were studied. CARP protein and mRNA levels were significantly increased by ISO (CARP/CSQ 161±10% of control, n=4, p<0.05; CARP mRNA/GAPDH mRNA 148±10% of control, n=9; p<0.05) and PE (CARP/CSQ 172±7%, n=4, p<0.05; CARP mRNA/GAPDH mRNA 178±20%, n=11, p<0.05). The effects of ISO and PE on CARP mRNA expression were completely reversed by the ERK inhibitor U0126 (p<0.05) but not by the p38 kinase inhibitor SB203580. Interestingly, CARP mRNA expression was not restricted to myocytes. CARP mRNA was much more abundant in isolated myocytes than in non-myocyte cells (ratio 6.6:1, p<0.05).

We claim:

1. Use of an inhibitor or antagonist of CARP protein or CARP mRNA for the manufacture of a medicament which can be used for the treatment of heart diseases.

2. Use according to claim 1, wherein the heart disease is heart failure or heart hypertrophy.

3. Use according to claim 2 wherein the heart hypertrophy is induced by isoprenalin or phenylephrine.

4. Use according to claim 1, wherein the inhibitor is capable of binding to CARP protein and/or CARP mRNA.

5. Use according to claim 1, wherein the inhibitor prevents CARP protein from binding to YB-1.

6. Use according to claim 1, wherein the inhibitor prevents CARP/YB-1 complex from binding to HF-1a/HF1-b/Mef-2 DNA elements.

7. Use according to claim 1, wherein the inhibitor prevents CARP protein from down-regulation of ANF or TNC from cardiac origin.

8. Use according to claim 1, wherein the inhibitor is capable of enhancing the expression and/or secretion of ANF from the heart.

9. Pharmaceutical composition applicable for the treatment of heart diseases comprising a substance having at least one of the following biological properties:
(i) inhibition CARP protein and/or CARP mRNA,
(ii) binding to CARP protein and/or CARP mRNA,
(iii) prevention of CARP from binding to YB-1, or of CARP/YB-1 complex from binding to HF-1a/HF1-b/Mef-2 DNA elements,
(iv) prevention of CARP protein from down-regulation of cardiac ANF or TNC,
(v) enhancement of the expression and/or secretion of ANF from the heart.

10. Use of CARP protein or CARP mRNA/DNA for screening of substances which block or inhibit CARP or CARP expression.

11. Use of CARP protein or CARP mRNA/DNA as negative regulator of the expression of alpha-myosin heavy chain mRNA.

12. Method for screening an inhibitor of CARP protein and/or CARP mRNA, the method comprising the following steps
(i) constructing a first expression vector comprising full-length human CARP cDNA together with a strong promoter and a selection marker,
(ii) constructing a second expression vector comprising a reporter gene which is under the control of HF-1a/HF1b/Mef-2 together with a selection marker which is different from that of the first expression vector
(iii) transfecting said first and said second expression vector into a suitable eukaryotic expression cell line, and
(iv) culturing transfected cells expressing CARP protein as well the reporter gene product which indicates the CARP activity, together with said inhibitor and measuring the reduced activity of said reporter gene product.

13. Use of CARP or modifications or variants of CARP having the biological properties of CARP as diagnostic means for detecting in vitro heart disorders like heart failure.

14. Method of treating heart diseases, wherein the method comprises administering to a patient suffering from heart failure a pharmaceutical composition as characterized in claim 9 in an amount, which is effective for a specific heart disease.

15. A method for screening an inhibitor of CARP protein comprising
(i) providing a recombinant CARP protein,
(ii) providing a recombinant YB-1 protein
together with an inhibitor and measuring the interference with the CARP protein/YB-1 complex formation.

16. A method for screening an inhibitor of CARP protein comprising
(i) providing a recombinant CARP/YB-1 protein complex,
(ii) providing a recombinant HF-1a/HF-1b/Met-2 DNA elements together with an inhibitor and measuring the interference with the CARP protein/YB-1 complex and the HF-1a/HF-1b/Met-2 DNA elements.

17. Use of the inhibitor U0126 for the manufacture of a medicament for the inhibition of CARP protein complex.

18. Use of claim 17 for the treatment of heart diseases.
